(19) United States
(12) Patent Application Publication
(10) Pub. No.: US 2020/0318059 A1
(43) Pub. Date: Oct. 8, 2020

(54) AUTOMATIC CULTURE SYSTEM, AND CELL CULTURE METHOD

(71) Applicant: HITACHI, LTD., Tokyo (JP)

(72) Inventors: Masashi MARUYAMA, Tokyo (JP); Masaki MATSUMORI, Tokyo (JP)

(21) Appl. No.: 16/754,430

(22) PCT Filed: Oct. 18, 2018
(86) PCT No.: PCT/JP2018/038761

§ 371 Date: Apr. 8, 2020

(30) Foreign Application Priority Data
Nov. 17, 2017 (JP) 2017-221926

(51) Int. Cl. C12M 1/36; C12M 1/34; C12N 9/50

(52) U.S. Cl. CPC C12M 41/48; C12M 41/26; C12N 9/50; C12M 41/28; C12M 41/16

(57) ABSTRACT

The purpose of the present invention is to provide an automatic culture system whereby cells can be released from a vessel by a simple constitution and damage on the cells can be minimized In the automatic culture system according to the present invention, cells are cultured in a stimulus-responsive culture vessel and then the cells are released from the culture vessel by stimulating the culture vessel with a solution which is supplied from another vessel (see FIG. 3).

### TECHNICAL FIELD

The present invention relates to an automatic culture system for culturing cells.

### BACKGROUND ART

Various types of automatic culture systems are developed for mass production of cells with stable quality. For example, the development of culture environment control, in which a large merit is obtained by machine control, has been advanced, and stabilization of cell quality can be expected. However, it is not necessarily easy to replace all works hitherto performed by a human operator with machines in cell culture.

Attempts have been made to reproduce human works by an automatic culture system equipped with many movable mechanisms such as a robot arm in a sterile space (PTL 1 to 3). Meanwhile, closed automatic culture systems constituted by closed flow paths (flow paths maintained air-tight) are generally simple and small in their structure compared to systems equipped with many movable mechanisms as in PTL 1 to 3. Further, a culture system that can release cells from a vessel without using a release solution such as an enzyme has been also developed (PTL 4).

### CITATION LIST

### Patent Literature

PTL 1: JP 2016-013061 A

PTL 2: WO 2016/147898 A

PTL 3: JP 2010-136666 A

PTL 4: WO 2013/116421 A

### SUMMARY OF INVENTION

### Technical Problem

The automatic culture systems as described in PTL 1 to 3 have problems that: (a) the system becomes large due to necessity for covering a large part of the system by a sterile space; (b) the mechanical mechanism is complicated; (c) production at low cost is difficult because a large number of members are required; (d) there is difficulty in maintenance; and the like.

In the closed automatic culture systems constituted by a closed flow path, which uses a release solution for releasing cells from a vessel, the cells need to be released from the release solution by using, for example, a centrifuge in order to minimize damage on the cells. The centrifuge is a system that is different from the automatic culture system and is not directly connected to the closed flow path. Therefore, a step of introducing cells into the centrifuge is separately required. The automation of this step is generally difficult because a complicated mechanism is required.

As in PTL 4, in the system that releases cells from a vessel without using a release solution, a complicated mechanism is generally required in order to release cells in the state capable of being subcultured. Thus, it is considered difficult to realize a simple system constitution.

The present invention is conceived in light of the problem described above, and an object of the present invention is to provide an automatic culture system whereby cells can be released from a vessel by a simple constitution and damage on the cells can be minimized.

### Solution to Problem

In the automatic culture system according to the present invention, cells are cultured by a culture vessel having stimulation responsiveness, and then the cells are released from the culture vessel by applying stimulation to the culture vessel with a solution supplied from another vessel.

### Advantageous Effects of Invention

According to the automatic culture system of the present invention, cells can be released from a vessel by a simple constitution and damage on the cells can be minimized.

### BRIEF DESCRIPTION OF DRAWINGS

FIG. 1 is a schematic view illustrating a step of subculturing cells using an enzyme.

FIG. 2 is a schematic view illustrating a step of subculturing cells using a stimulation response.

FIG. 3 is a constitution view of the automatic culture system 100 according to Embodiment 1.

FIG. 4 is a specific example of the constitution of an automation mechanism.

### DESCRIPTION OF EMBODIMENTS

### <General Step of Cell Culture>

In cell subculture, cells are generally released from a vessel with, for example, enzymes, which decompose an extra cellular matrix (ECM), such as protease (e.g., trypsin, accutase, collagenase, natural protease, chymotrypsin, elastase, papain, pronase, or a recombinant thereof), and chelate agents which release a complex of ECM by being coordinated to an alkaline earth metal ion at three or more sites (for example, ethylenediaminetetraacetic acid (EDTA)). The enzyme or the chelate agent remains in the cell suspension after releasing the cells, and this damages the cells. Therefore, the necessity to separate the enzyme or the chelate agent from the cells by, for example, a centrifuge becomes. Centrifugation, which generally involves a step of transferring a cell suspension from a culture vessel to a vessel for centrifugation, requires a complicated mechanism for automation by a machine.

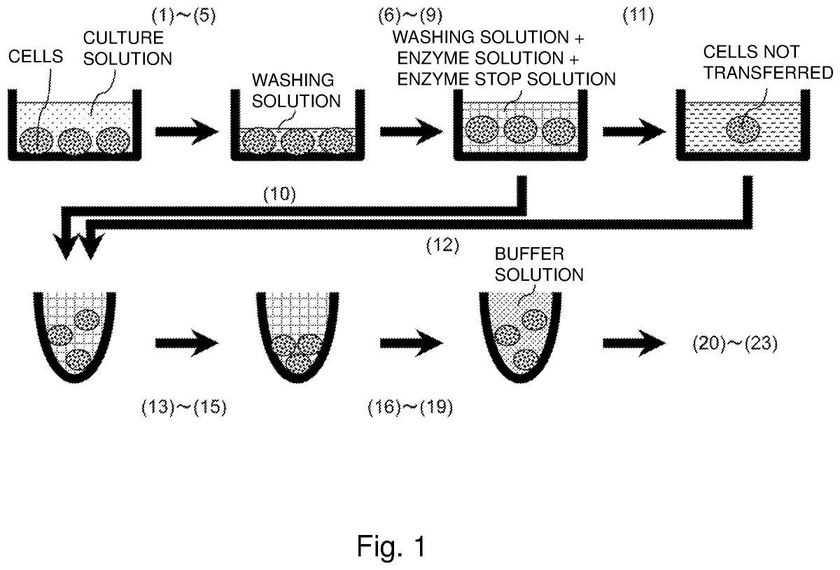

FIG. 1 is a schematic view illustrating a step of subculturing cells using an enzyme. This case includes the following steps: (1) removing a culture solution; (2) adding a washing solution; (3) removing the washing solution; (4) adding a washing solution; (5) removing the washing solution; (6) adding an enzyme solution; (7) checking cell release; (8) adding an enzyme stop solution; (9) applying a liquid flow to cells; (10) transferring a cell suspension to a centrifuge tube; (11) adding a washing solution; (12) additionally transferring a cell suspension to the centrifuge tube; (13) closing the lid of the centrifuge tube; (14) setting the centrifuge tube to a centrifuge; (15) performing centrifugation; (16) opening the lid of the centrifuge tube; (17) removing a supernatant; (18) adding a buffer solution; (19) applying a liquid flow to a cell mass; (20) collecting the cell suspension from the centrifuge tube; (21) counting the number of cells; (22) measuring the amount of cell suspension required; and (23) performing dissemination. In this example, for example, the steps (10), (12), (13), (14), (16), (19), and (20) are required for performing centrifugation, and these steps require a mechanically complicated mechanism.

The closed automatic culture system is a culture system in which flow paths between the vessels are constituted by air-tight flow paths. The closed automatic culture system has the following advantages: (a) the risk of contamination is small because the culture system does not contact the outside air after setting; (b) liquid can be simply sent by a tube without using a mechanism such as a pipette; (c) it is not necessary to provide the system in a sterile space unlike the automatic culture system using a robot arm; and the like. In this type of automatic culture system, a culturing step is performed by transferring a solution between vessels, and therefore the structure is simple. Note that the release solution needs to be separated similarly to FIG. 1, and therefore a complicated mechanism is required similarly to FIG. 1 in order to completely automate the step.

In FIG. 1 and the closed automatic culture system, a complicated mechanism is required. This is because a release solution damages the cells and there is a need to remove the release solution. Thus, the present invention proposes a constitution that does not use a complicated mechanism for transferring cells to a centrifuge by releasing cells from a vessel without using a release solution. By this, automation of cell subculture can be achieved with only a simple mechanism utilizing advantages due to a simple structure of the closed automatic culture system.

### Embodiment 1

As a means for releasing cells from a vessel without using components that damage cells (i.e., a release solution), a stimulus-responsive culture vessel is exemplified. The stimulus-responsive culture vessel is a culture vessel in which the physical properties of the surface of the culture vessel are changed by a physical and chemical stimulation, and thereby adhesiveness to cells may be changed. Examples of the stimulation include a temperature change, a pH change, a solution composition change (e.g., an ion concentration change, a sugar concentration change, a protein concentration change, and a biotin concentration change), addition of an oxidizing agent, and addition of a reducing agent, but are not limited thereto.

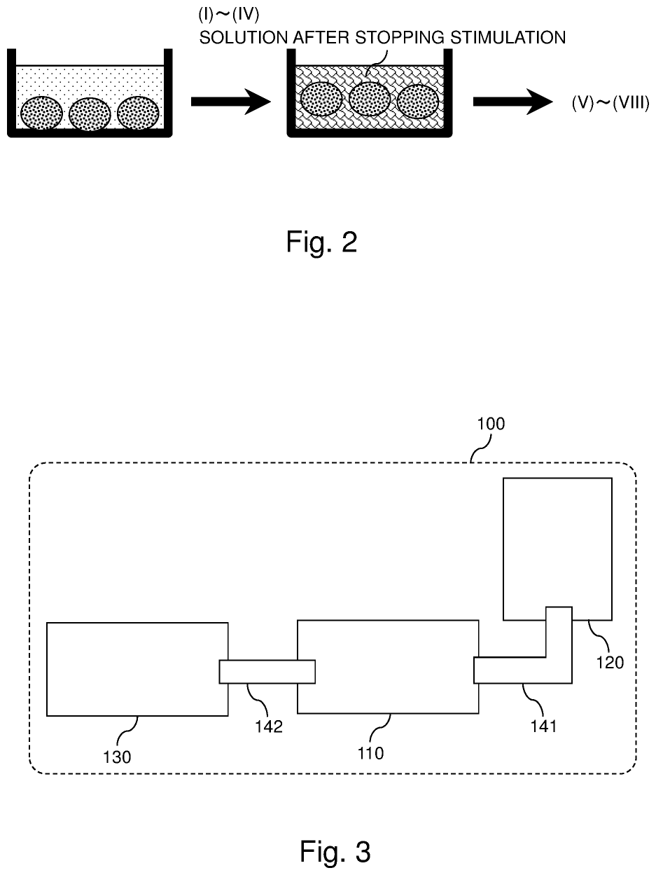

FIG. 2 is a schematic view illustrating a step of subculturing cells using a stimulation response. This case includes the following steps: (I) removing a culture solution; (II) applying a stimulation; (III) stopping the stimulation, (IV) applying a liquid flow to cells; (V) collecting a cell suspension from a culture vessel; (VI) counting the number of cells; (VII) measuring the amount of cell suspension required; and (VIII) performing dissemination. Cell subculture can be performed with shorter steps compared to FIG. 1.

In a case where the temperature change is used as the stimulation applied to the stimulus-responsive vessel, cells are cultured on the temperature-responsive culture vessel, and then, the cells are released by allowing a culture medium or a buffer solution having a temperature that is different from the culture temperature to act, and thus decreasing adhesiveness to the cells. In animal cells, a temperature change within a range of 4° C. to 37° C. does not adversely affect cells within a short period of time in many cases. When stimulation is stopped, a solution of approximately culture temperature may be added.

In a case where cells are released using a culture medium of a low temperature, cells are released by supplying a solution of approximately ambient temperature, resulting in a component constitution of the cell suspension that allows cells to survive for a long period of time. Thus, the component of the solution need not to be further adjusted. Note that, for example, in a case where such an adjustment is necessary for analysis, the component adjustment may be further performed according to application. In a case where cells are released by allowing a buffer solution of a low temperature to act, it is desirable to adjust to a component constitution that allows cells to survive for a long period of time by further adding a culture medium or a concentrated culture medium.

An example of the temperature-responsive culture vessel is one including a temperature-responsive material on the surface of the culture vessel. Examples of the temperature-responsive material include: (a) polyacrylamide derivatives such as poly (N-isopropyl acrylamide) and poly(N,N-diethylacrylamide); (b) polymethacrylate derivatives such as poly(oligoethylene glycol methacrylate); (c) polyvinyl ether derivatives such as poly(methyl vinyl ether); (d) polyvinylamine derivatives such as poly(N-vinylcaprolactone); (e) polyalkylene glycol derivatives such as a polyethylene glycol copolymer or a polypropylene glycol copolymer; (f) polyoxazoline derivatives such as poly(oxazoline); (g) polysaccharide derivatives such as hydroxypropyl cellulose; (h) polypeptide; and (i) polypeptide derivatives, but are not limited thereto. The shape of the vessel is not limited.

In a case where the pH change is used as the stimulation applied to the stimulus-responsive vessel, cells are cultured on the pH-responsive culture vessel, and then, the cells are released by allowing a culture medium or a buffer solution with a pH that is different from the normal culture condition to act, and thus decreasing adhesiveness to the cells. Although it depends on the type of cell, a minute pH change does not adversely affect cells within a short period of time. When stimulation is stopped, pH may be neutralized or diluted by addition of a culture medium and a buffer solution.

In a case where a solution with a pH that is different from the normal culture condition is allowed to act, it is desirable to adjust to a component constitution that allows cells to survive for a long period of time by adding a culture medium or a concentrated culture medium after releasing cells. Although the direction of the pH change may be both on the acidic side or the alkali side, a direction that less adversely affects cells is desirable.

An example of the pH-responsive culture vessel is one including a pH-responsive material on the surface of the culture vessel. Examples of the pH-responsive material include (a) polyamines such as polylysine, polyethyleneimine, and chitosan; (b) amine-containing temperature-responsive polymers such as a poly(diethylaminoethyl methacrylate)-poly(N-isopropylacrylamide) copolymer or a polypropylene glycol-ethylenediamine copolymer; (c) polycarboxylic acids such as polyglutamic acid and carboxymethyl cellulose; (d) carboxylic acid-containing temperature-responsive polymers such as a poly(acrylic acid)-poly(N-isopropyl acrylamide) copolymer; (e) polypeptide; and (f) adhesive proteins, but are not limited thereto. The shape of the vessel is not limited.

For cells, cell membranes, and ECMs that have sufficient responsiveness to pH, cell adhesion sometimes be decreased to a level that can release cells by weak acid or the like. In a case where such cells are used, the pH-responsive culture vessel is not necessarily used.

FIG. 3 is a constitution view of the automatic culture system 100 according to Embodiment 1. The automatic culture system 100 includes a culture vessel 110, a solution vessel 120, and a released cell vessel 130. The culture vessel 110 and the solution vessel 120 are connected by a first closed flow path 141, and the culture vessel 110 and the released cell vessel 130 are connected by a second closed flow path 142.

The culture vessel 110 is the above-described stimulus-responsive vessel and used for cell culture. The solution vessel 120 accommodates a solution that applies stimulation to the culture vessel 110 and thereby decreases adhesiveness to cells. Since damage on the cells caused by this solution needs to be less, desirably, the concentration of the enzyme or the chelate agent that is conventionally used for releasing cells is sufficiently low. Specifically, the concentration of the enzyme or the chelate agent in the solution is desirably less than 0.01 wt %. This is because the concentration at this level is targeted when the enzyme or the chelate agent is separated in centrifugation. The released cell vessel 130 accommodates cells released from the culture vessel 110.

In a case where the automatic culture system 100 is configured as a closed culture system, the culture system is configured so that the first closed flow path 141 and the second closed flow path 142 have an air-tight structure, and cells are not exposed to the outside air when the solution passes through these flow paths. By such a configuration, the effect of the present invention can be exerted while the advantages of the closed culture system can be utilized. Further, at least any of the respective vessels may be configured to be air-tight.

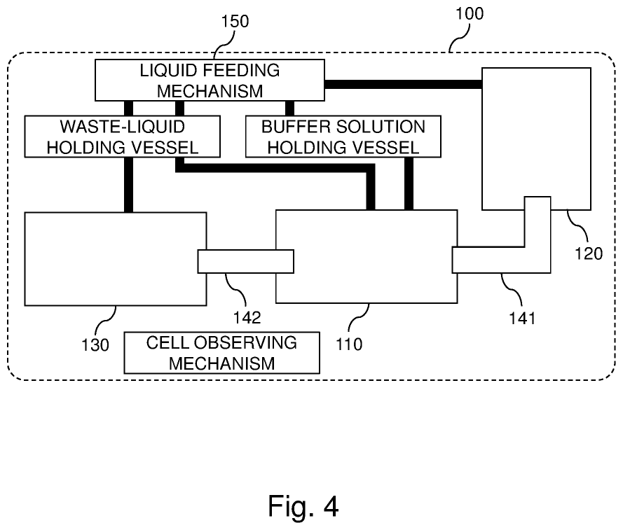

FIG. 4 is a specific example of the constitution of an automation mechanism. Respective steps of cell subculture can be automatically performed by providing a liquid feeding mechanism 150, a buffer solution holding vessel, a waste-liquid holding vessel, and a cell observing mechanism in addition to the constitution described in FIG. 3, and transferring the solution between respective vessels by the liquid feeding mechanism 150.

The steps (I) to (VIII) of the example described above can be implemented, for example, as follows. The step (I) can be implemented by transferring the culture medium in the culture vessel 110 to the waste-liquid holding vessel by the liquid feeding mechanism 150. The step (II) can be implemented by transferring the stimulation solution in the solution vessel 120 to the culture vessel 110 by the liquid feeding mechanism 150. The step (III) can be implemented by transferring a buffer solution from the buffer solution holding vessel to the culture vessel 110 by the liquid feeding mechanism 150. The step (IV) can be implemented by generating a liquid flow in the step (III). The steps (V) to (VIII) can be implemented by counting the number of cells using the cell observing mechanism and transferring a certain amount of cell suspension in the culture vessel 110 to the released cell vessel 130 by the liquid feeding mechanism 150.

### Embodiment 1: Summary

In the automatic culture system 100 according to Embodiment 1, cells are cultured using a stimulus-responsive vessel, and then released from a vessel by transferring a solution for applying stimulation. This eliminates the necessity of using a solution that damages cells, such as a release solution. It is therefore not necessary to transfer the cells to the outer system such as a centrifuge. Accordingly, cell subculture can be automated by a simple system constitution.

### Embodiment 2

As the stimulation applied to the stimulus-responsive vessel, an ion concentration change can be used. In this case, cells can be cultured on the ion-responsive culture vessel and released by the ion concentration change. Even if the ion concentration in the culture condition is changed for cell releasing, such a change does not adversely affect the cells within a short period of time. When stimulation is stopped, the ion concentration may be adjusted by using an addition solution or a diluent.

Some of ECMs represented by cadherin have alkaline earth metal ion responsiveness. For such ECMs, cell adhesion is sometimes decreased to a level that can release cells by decreasing the alkaline earth metal ion concentration. In a case where such cells are used, the ion-responsive culture vessel is not necessarily used.

As the stimulation applied to the stimulus-responsive vessel, a change in the concentration of the component that causes no problem when the component coexists with cells in the culturing or the component contained in the culture medium can be used. Examples of such a component include glucose (stimulates against a glucose-responsive material such as boronic acid or lectin), biotin (stimulates against a biotin-responsive material such as avidin or streptavidin), polyethylene glycol (PEG) (stimulates against a PEG-responsive material such as an anti-PEG antibody), and glutathione (reduction stimulation against a readily reducible bond such as a disulfide bond).

Instead of applying a stimulation by allowing a solution to act, for example, a stimulation that does not require solution exchange can be used, such as (a) changing temperature by taking out a culture vessel from an incubator and exposing the culture vessel to the outside air; (b) irradiating light; and (c) applying mechanical stimulation. In this case, the culture solution is not necessarily removed prior to application of the stimulation, and therefore this is advantageous in simplicity of the process. On the other hand, a special mechanism that corresponds to the stimulation is required as a stimulation application mechanism, and therefore it is not advantageous in view of simplicity of the system constitution compared to a method of releasing cells by only allowing a solution to act.

Note that, in a case where cells are released by allowing a solution to act, the osmotic pressure of the solution to act desirably does not exceed a range of osmotic pressure that does not adversely affect cells. The range of osmotic pressure that does not adversely affect cells is typically 260 to 320 mOSM/kg.

### Regarding Modified Example of the Present Invention

The present invention is not limited to the above-described embodiments, and various modification examples are included. For example, the above-described embodiments have been described in detail for easy understanding of the present invention and does not necessarily include all the configurations described above. In addition, a part of a configuration of an embodiment may be replaced with a configuration of another embodiment. Further, a configuration of an embodiment may be added to a configuration of another embodiment. In addition, regarding a part of the configuration of each of the embodiments, addition of another configuration, deletion, or replacement can be made.

In a case where cell subculture is performed using the automatic culture system 100 according to the present invention, although depending on the properties of cells to be cultured, released cells need to be a state capable of being disseminated. Therefore, cells are preferably released in a state of a single cell or a small clamp. In general, if cells are released before being confluent, cells can be released without being a large clamp. For example, if sites of the culture vessel, which are in contact with cells, are patterned in an appropriate morphology, released cells can be easily obtained in a state where the morphology of the cells is a single cell or a small clamp. In addition to the physical patterning such as irregularity, chemical patterning such as introduction of cell non-adhesive components or release of cell adhesive components may be employed. In a case where the sites in contact with cells are porous, fibrous, or bead-like, there is a possibility that formation of the large clamp can be minimized by controlling the area or curvature per site.

The stimulus-responsive material of the culture vessel 110 may be adhesive proteins, derivatives of adhesive proteins, and recombinants of adhesive proteins. In a case where a response of the adhesive protein to the stimulation changes the structure of the adhesive protein or decreases the degree of polymerization of the adhesive protein, thus decreasing adhesiveness, the adhesive protein can be used as a stimulus-responsive material.

In addition to the constitution described in FIGS. 3 and 4, the automatic culture system 100 may include mechanisms such as a culture medium holding vessel, a system control mechanism, a network connection mechanism, a reagent holding vessel, a gas concentration control mechanism, a temperature control mechanism, a sterilizing mechanism, a vessel transfer mechanism, sensors, a sampling mechanism, and a light irradiation mechanism; and may include additional various types of vessels and mechanisms. A plurality of automatic culture mechanisms and automatic subculture mechanisms may be provided inside one housing.

The automatic culture system 100 may be used for culturing adhesive cells. Floating cells can be subcultured by only diluting a cell suspension due to its original properties, and therefore the effect by the automatic culture system 100 is small.

The types of adhesive cells that are cultured by the automatic culture system 100 are not limited. The origin of cells (e.g., primary cells, cultured cells, established cell lines, and gene-recombined cells) and species are not limited. Examples of the adhesive cell include: stem cells (e.g., mesenchymal stem cells, ES cells, and iPS cells); epithelial cells (e.g., vascular epithelial cells, and bile duct epithelial cells); endothelial cells (e.g., vascular inner non cells, and lymphatic endothelial cells); fibroblasts (e.g., NIH3T3); hepatic cells; pancreatic islet cells; nerve cells; myocardial cells; myogenic cells; cancer cells; macrophage; HeLa cells; and CHO cells. Cells to be cultured need not to be one type, and may be applied to a co-culture system.

In a case where adhesive cells have differentiation potential, cells may also be differentiated during culturing using the automatic culture system 100. In a case where responses of differentiated cells and undifferentiated cells to stimulation are different, differentiated cells and undifferentiated cells can be separated during the step of releasing cells by the stimulation response depending on the condition. In this case, cells can be simply purified by, for example, transferring only cells required to be collected to the next step. Even in a case where adhesive cells having differentiation potential are grown while the undifferentiated state of the adhesive cells is maintained, in the case where responses of differentiated cells and non-differentiated cells to stimulation are different, only undifferentiated cells can be transferred to the next step depending on the condition. A case where the adhesiveness of cells is decreased during culturing is the same. For example, this applies to a case where cells are transformed into floating cells due to cell differentiation or adaptation, or a case where formation of aggregates such as spheroid decreases adhesiveness.

In a case where cultured cells are collected, it is not always necessary to release cells by a method similar to the cell subculture. It is also not necessary for culturing at a stage prior to collection to use a culture vessel having stimulation responsiveness. In cell collection, the morphology of the cell is not particularly limited and may be, for example, a single cell, a small clamp, a large clamp, or a sheet-like cell. Cell collection may be performed outside the automatic culture system 100. The automatic culture system 100 may be used for not collecting cells themselves, but for continuously collecting components secreted by cells.

### EXAMPLES

Hereinafter, the results of verification of the effects of the automatic culture system 100 according to the present invention will be described along with Comparative Examples.

### Example 1

Cell release using stimulation with pH and solution composition change was studied by using a pH-responsive culture vessel. The pH-responsive culture vessel was produced as described in JP 2015-117114, hMSCs (Takara Bio Inc.) were disseminated and cultured for five days in accordance with the protocol recommended by Takara Bio Inc. A culture solution was removed from the vessel including the obtained hMSCs, after which phosphate buffered saline (PBS) with a pH of 6.5 was added to the vessel, and the hMSCs were incubated. Thereafter, the stimulation with pH and solution composition change was stopped by addition of a culture medium of 37° C., and a liquid flow was generated to further release the hMSCs from the bottom of the vessel. The obtained cell suspension was transferred to another vessel, and the number of cells and the survival ratio were counted. The number of cells remaining in the bottom of the vessel was also counted, and the release ratio was calculated. As a result, the survival ratio of cells in the cell suspension obtained by the release using stimulation with pH and solution composition change was 90%, and the release ratio was 95%, which were sufficiently high values.

### Example 2

The cells obtained in Example 1 were evaluated. The cell suspension obtained in Example 1 was disseminated in a new vessel and cultured for five days in the same manner. As a result, the growth ratio and the survival ratio of the obtained cells were equivalent to those of Example 1, and a decrease in the proliferative ability of cells due to release using stimulation with pH and solution composition change was not observed.

### Example 3

Cell release using stimulation with pH and solution composition change was studied by using a pH-responsive culture vessel. Chinese hamster ovary (CHO) cells were cultured and subcultured in the same manner as in Example 1. As a result, the survival ratio of cells in the cell suspension obtained by the release using stimulation with pH and solution composition change was 90%, and the release ratio was 90%, which were sufficiently high values. The proliferative ability after subculture was almost equivalent to the proliferative ability before subculture.

### Example 4

Cell release using stimulation with temperature and solution composition change was studied by using a temperature-responsive culture vessel. hMSCs were cultured and subcultured in the same manner as in Example 1 using RepCell, available from CellSeed Inc. as a temperature-responsive culture vessel and using PBS of 10° C. as a cell release solution. As a result, the survival ratio of cells in the cell suspension obtained by the release using stimulation with temperature and solution composition change was 85%, and the release ratio was 90%, which were sufficiently high values. The proliferative ability after subculture was almost equivalent to the proliferative ability before subculture.

### Comparative Example 1

As Comparative Example 1, cell release was studied using a normal polystyrene culture vessel. hMSCs were disseminated in a normal polystyrene culture vessel, cultured in the same manner as in Example 1, and studied in the same manner as in Example 1. The results demonstrate that cells were released from the polystyrene culture vessel by only 5% or less by the stimulation with pH and solution composition change under the condition of a gentle liquid flow.

### Comparative Example 2

As Comparative Example 2, cell release from a polystyrene culture vessel using a strong liquid flow was studied. hMSCs were cultured in the same manner as in Comparative Example 1, and cells were released from the bottom of the culture vessel by spraying a culture medium to the obtained cells at a high flow rate for 30 seconds. As a result, the survival ratio of cells in the obtained cell suspension was 75%, which was a low value.

### Comparative Example 3

As Comparative Example 3, the effects on cells were studied in a case where centrifugation was not performed by a conventional cell release method. CHO was cultured on a normal polystyrene culture vessel, and a cell suspension obtained by releasing cells using a trypsin/EDTA solution was disseminated in a polystyrene culture vessel without performing a treatment of stopping enzymes or removing enzymes or chelate agents by centrifugation. Then, the proliferative ability of cells after dissemination was measured. As a result, the proliferative ability was decreased by 20% or more in a case where dissemination was performed without centrifugation compared to a case where centrifugation was performed.

### REFERENCE SIGNS LIST

100 automatic culture system
110 culture vessel
120 solution vessel
130 released cell vessel
141 first closed flow path
142 second closed flow path
150 liquid feeding mechanism

1. An automatic culture system for culturing cells, the automatic culture system comprising:
a culture vessel that cultures the cells; and
a solution vessel that accommodates a solution to be supplied to the culture vessel, wherein
the culture vessel has stimulation responsiveness in which adhesiveness to the cells changes due to a stimulation applied by the solution.

2. The automatic culture system according to claim 1, wherein
in the solution, a concentration of an enzyme for reducing a degree of polymerization of an extracellular matrix is less than 0.01 wt %; and
in the solution, a concentration of a chelate agent which can be coordinated to an alkaline earth metal ion is less than 0.01 wt %.

3. The automatic culture system according to claim 2, wherein
the enzyme for reducing a degree of polymerization of an extracellular matrix is protease; and
the chelate agent which can be coordinated to an alkaline earth metal ion is a chelate agent which can be coordinated to an alkaline earth metal ion at three or more sites.

4. The automatic culture system according to claim 3, wherein
the protease is trypsin, accutase, collagenase, natural protease, chymotrypsin, elastase, papain, pronase, or a recombinant thereof; and
the chelate agent which can be coordinated to an alkaline earth metal ion at three or more sites is ethylenediaminetetraacetic acid.

5. The automatic culture system according to claim 1, wherein
the culture vessel has stimulation responsiveness in which adhesiveness to the cells changes due to a temperature change;
the culture vessel is formed using, as a stimulus-responsive material, at least any of polyacrylamide derivatives, polymethacrylate derivatives, polyvinyl ether derivatives, polyvinylamine derivatives, polyalkylene glycol derivatives, polyoxazoline derivatives, polysaccharide derivatives, polypeptide, and polypeptide derivatives.

6. The automatic culture system according to claim 1, wherein the culture vessel has stimulation responsiveness in which adhesiveness to the cells changes due to a pH change;
the culture vessel is formed using, as a stimulus-responsive material, at least any of polyamines, amine-containing temperature-responsive polymers, polycarboxylic acid, carboxylic acid-containing temperature-responsive polymers, polypeptide, and adhesive proteins.

7. The automatic culture system according to claim 1, wherein the culture vessel has stimulation responsiveness in which adhesiveness to the cells changes due to a stimulation applied by at least any of a change in a composition of the solution, oxidization reaction, and reduction reaction.

8. The automatic culture system according to claim 1, further comprising a liquid feeding mechanism that transfers the solution from the solution vessel to the culture vessel.

9. The automatic culture system according to claim 1, further comprising:
a released cell vessel that accommodates the cells released from the culture vessel;
a first flow path that connects the solution vessel and the culture vessel; and
a second flow path that connects the culture vessel and the released cell vessel, wherein the first flow path and the second flow path each have an air-tight structure.

10. The automatic culture system according to claim 9, wherein at least any of the culture vessel, the solution vessel, or the released cell vessel has an air-tight structure.

11. A cell culture method, wherein cells are cultured by using the automatic culture system according to claim 1.

12. The cell culture method according to claim 11, the method comprising: releasing the cells from the culture vessel by transferring the solution from the solution vessel to the culture vessel by using the liquid feeding mechanism provided in the automatic culture system, and thus decreasing adhesiveness of the cells to the culture vessel.
